(19) United States
(12) Patent Application Publication
(10) Pub. No.: US 2026/0305669 A1
(43) Pub. Date: Oct. 8, 2026

(54) ORAL DOSE COMPOSITIONS OF TRIBUTYRIN FOR THE GENERATION OF BUTYRATE IN THE GUT

(71) Applicant: NutriScience Innovations LLC, Milford, CT (US)

(72) Inventor: Michael Lelah, Arlington Heights, IL (US)

(21) Appl. No.: 19/460,647

(22) Filed: Jan. 27, 2026

Related U.S. Application Data
(63) Continuation of application No. 17/674,885, filed on Feb. 18, 2022.
(60) Provisional application No. 63/152,389, filed on Feb. 23, 2021.

(51) Int. Cl. A23L 33/12; A23L 33/135; A23L 33/21

(52) U.S. Cl. CPC A23L 33/12; A23L 33/135; A23L 33/21; A23V 2400/175

(57) ABSTRACT

The present disclosure relates generally to compositions for a dietary supplement, a food supplement, and/or a nutritional supplement, useful in the generation of butyrate in the gastrointestinal track or gut of a mammal for effecting and improving health and wellness. The present disclosure also relates to the use of a dietary supplement provided in a therapeutically effective amount as a replacement for probiotics and prebiotic usage. Additionally, the present disclosure relates to a dietary supplement composition including a low therapeutic dose of tributyrin either alone, or in combination with prebiotics and/or probiotics to enhance health and wellness. Furthermore, the present disclosure also relates to a low dose dietary supplement composition which generates therapeutic amounts of butyrate in the gut of a mammal independent of the state of the microbiome to achieve desired health benefits of butyrate.

### TECHNICAL FIELD

The present disclosure relates generally to compositions for a dietary supplement, also known as a food supplement, or as a nutritional supplement, useful in the generation of butyrate in the gastrointestinal tract or gut of a mammal for affecting and improving health and wellness. The present disclosure also relates to the use of a dietary supplement provided in a therapeutically effective amount as a replacement for probiotics and prebiotic usage. Additionally, the present disclosure relates to a dietary supplement composition having a low therapeutic dose of tributyrin either alone, or in combination with prebiotics and/or probiotics to enhance health and wellness. Furthermore, the present disclosure also relates to a low dose supplement and/or composition which generates therapeutic amounts of butyrate in the gastrointestinal tract or gut of a mammal independent of the state of the microbiome of the mammal to achieve the desired health benefits of butyrate.

The present disclosure further relates to a powder form composition of tributyrin as a dietary supplement for providing therapeutic generation of butyrate. The powder composition has a low water activity making it suitable for combining with probiotics and other moisture sensitive dietary ingredients.

### BACKGROUND

The role of the gut microbiome is the focus of studies on how the microbiome impacts the overall health and wellness of both humans and animals, not only as it relates to the digestive system, but also the link between mood and/or cognition—the “gut-brain connection.” With the goal to achieve a “healthier” microbiome and an overall healthier state, many people (and animals) consume oral prebiotics and prebiotics. Orally consumed probiotics and prebiotics have been shown to be absorbed by the gut and help generate short chain fatty acids (SCFAs) including acetate, propionate and butyrate, which have been shown to be responsible for providing energy to the colonocytes and also to act as signaling molecules. Butyrate is the SCFA of most interest in effecting and improving health. Butyrate is also called a postbiotic, being one of the metabolites of live bacteria. Butyrate is a signaling molecule involved with improving health and wellness related to gut health, cognitive health, metabolic health, insulin sensitivity, cardiovascular health, pancreatic health, liver health, skin health, immune health, and in fact the health of almost all body organs; butyrate receptors are found in almost all cells in the body.

Probiotics are microorganisms which are intended to have health benefits when consumed. Probiotics include bacteria and yeast which are edible and are orally consumed. Common probiotics include various strains of lactobacilli and bifidobacteria. New research into other types of bacteria for use as probiotics include bacteriodes and clostridium genera. Probiotics also include spores which may be derived from plants, fungi or algae. Most probiotics produce SCFAs and specifically butyrate in the body, either directly or indirectly. For example, lactobacillus probiotics can generate lactate, which is then converted into butyrate. Probiotics include a new class of inactivated or dead probiotics, called parabiotics for which health benefits are currently being studied. It is not clear whether parabiotics can generate SCFAs including butyrate.

Prebiotics are compounds in foods which can induce growth or activity of beneficial microorganisms in the microbiome. Prebiotics can in this manner affect both exogenous microorganisms (probiotics) and also endogenous microorganisms which are present in the microflora of the gut, also referred to as the microbiome. Prebiotics can alter the composition of the microflora within the gut. Prebiotics are typically dietary fibers which may ferment and thus “feed” the microorganisms in the gut. While prebiotics are typically dietary fibers, included in the definition and scope of “prebiotics” are direct butyrate generators including tributyrin. Also included are bacteriophages, which are viruses that may be orally consumed to target and consume “bad bacteria” such as E. coli thereby allowing “good bacteria” such as bifidobacteria to colonize and grow in the gut. Prebiotics directly (in the case of tributyrin) or indirectly (as with most prebiotics) generate SCFAs and specifically butyrate in the gut. Prebiotics “feed” probiotics which can then generate the beneficial SCFAs, including butyrate.

Butyrate generation occurs in the gut microbiome. However, the state, condition or composition of the gut microbiome is dependent on a number of internal and environmental factors. These include the composition of the microbiome bacteria—the presence and amounts of good and bad bacteria, the state of the microbiome due to an infection and/or the use of antibiotics, the diet on a particular day, the overall state of health or disease of an individual human or animal at a particular moment in time, genetic factors, etc. These factors lead to variation between individuals and even within an individual for the ability of the diet and microbiome to generate butyrate. In fact, many studies using probiotics and/or prebiotics are dependent on utilizing larger sample sizes of individuals, in order to statistically demonstrate health benefits of such use. Not only is there variation between individuals in responses, but there will also be a number of non-responders where the use of probiotics and/or prebiotics has little or no effect.

Such variation in the gut microbiome also applies to the generation of butyrate. The advantage of a direct butyrate generator, such as tributyrin, is that therapeutic levels of butyrate generation is independent of the state, condition or composition of the microbiome of the individual. The composition of the microbiome, and/or the growth or reduction of different bacterial species in the microbiome, as measured by the composition of fecal matter, is irrelevant to the generation of therapeutic levels of butyrate by direct butyrate generators, such as tributyrin.

A potential issue with probiotics is that they typically need to be refrigerated or protected at room temperature and must be formulated with overages approaching or greater than 100% to assure potency at the end of shelf life. In addition, many of the probiotics in common use in dietary supplements are relatively poor butyrate generators. The problem with prebiotics is that they must be typically consumed in large quantities (typically 4-20 grams per day) in order to have a prebiotic effect; however, in some cases large doses will cause gut distress, bloating and gas when consumed at these levels due to their fermentation in the body which is necessary for their effect. A potential issue with combining prebiotics and probiotics (sometimes called pre/pro-biotics) is that the water activity of the prebiotic is too high. This high level of moisture in the combined product degrades the probiotic and makes formulating dietary supplements difficult.

An alternative to probiotics/prebiotics is the oral consumption of short chain fatty acids themselves, specifically butyrate, which mimic their generation in the gut. Sodium butyrate, butyric acid and other butyrate chemical entities generally have a very astringent taste, and also taste and smell like vomit, so are generally not suitable for human oral consumption. Tributyrin is also known chemically as glycerol tributyrate, glyceryl tributyrate or propane-1,2,3-triyl tributyrate. This food ingredient is known to be a source of butyrate as it is lysed by lipases in the small intestine into glycerol and butyrate. This action which involves an enzymatic reaction, slows the generation of butyrate in the gut, with lower amounts generated in the large intestine and more generated in the small intestine. This is preferential to the action of butyrate and thus is a positive feature of tributyrin. When reviewing pK curves of butyrate in plasma, it is noted that ingestion of sodium butyrate may take approximately 15-45 minutes to peak time in plasma, while tributyrin peak times are on the order of hours. This indicates that even if sodium butyrate or butyric acid were consumed (their taste profile is very poor for human oral consumption), they would be less suitable sources of butyrate.

Tributyrin is much more suitable because of its slower conversion to butyrate, and propensity for butyrate generated by tributyrin to be formed in the smaller intestines. Tributyrin is a food ingredient for use in dietary supplements and foods for the therapeutic effect of generating butyrate in the gut and is more suitable for human consumption than butyric acid or other types of butyrates. In commercial therapeutic dietary supplement applications, tributyrin is sometimes called a “postbiotic” but this is technically incorrect. Tributyrin is more correctly a “prebiotic” since in the body it generates butyrate, which is more correctly, the postbiotic.

Tributyrin is used a food additive and is also sold as a dietary supplement, sometimes in an encapsulated form. As a food additive, it is used for its physical and flavor characteristics only and not as a therapeutic agent. For example, tributyrin is used as a flavoring in butter and margarine. In dietary supplement commercial applications, dosages between 300 mg and 1,000 mg have been used. The science for tributyrin as a dietary supplement is primarily based on animal studies. Tributyrin has been evaluated for use in weight management and metabolic wellness, gut health, sleep, and immune system support.

In terms of gut health, there have been several studies on the potential use of tributyrin for improving gut health. Impaired gut-liver axis is a potential factor contributing to alcohol-related liver problems. Ethanol depletes intestinal integrity and causes gut dysbiosis, which is concerning since the gut-host mutualistic relationship is disrupted. Gut dysbiosis potentially jeopardizes benefits this relationship provides including metabolism, host immunity and barrier protection. Additionally, gut dysbiosis may lead to butyrate deficiency due to microbiota alterations.

Cresci et al. conducted a study to determine whether prophylactic tributyrin could protect the intestinal barrier and liver in mice during combined chronic-binge ethanol exposure. C57BL/6J mice exposed to ethanol-containing diet for 10 days, control mice were isocalorically pair-fed maltose dextrin for ethanol. Diets were supplemented (5 mM) with Tributyrin or glycerol.

Two important protective features of the intestinal barrier are mucus and tight-junction (TJ) protein complexes. Histopathology and immunohistochemistry results showed that ethanol exposure caused delocalization of TJ (i.e., leaky gut) in both the proximal colon and ileum. Tributyrin co-treatment maintained immunoreactive staining intensity of TJ proteins and co-localization of ZO-1 and occluding to similar patterns visualized in control mice. In addition, tributyrin influenced hepatic expression of Toll-like receptors and the inflammatory cytokine TNF-α, mitigated liver injury and induced a differential liver injury pattern in ethanol exposed mice. These data suggest that tributyrin may be useful for supporting a healthy gut lining and normal, healthy intestinal permeability.

As mentioned, butyrate is the preferential fuel of colonocytes, and it functions as a trophic, anti-inflammatory, pro-apoptotic and anti-carcinogenic agent. All these properties make butyrate a potential adjuvant in the treatment of inflammatory bowel dysbiosis, particularly ulcerative colitis (UC). Leonel A. et al. evaluated the efficacy and mechanisms of action of tributyrin supplementation in the prevention of mucosal damage in experimental colitis. Mice were divided into three groups. The control animals received water and the standard rodent diet. The colitis animals received a dextran sodium sulphate (DSS) solution and the standard diet. The colitis+tributyrin animals received a DSS solution and a tributyrin-supplemented standard diet (5 g/kg diet). The research determined mucosal damage and the activation of immune cells and cytokines, analyzed oxidative stress and assessed intestinal permeability.

Results showed that the animals that supplemented with tributyrin, compared with non-supplemented mice, presented an improved mucosal architecture (with more preserved areas), a reduced extension and intensity of the inflammatory infiltrate, and an absence of mucosal ulcerations. A lower level of hydroperoxide and higher levels of superoxide dismutase (SOD) and catalase activities were associated with tributyrin supplementation. In addition, tributyrin-supplemented mice showed reduced intestinal permeability to the levels intermediate between the control and colitis groups. These data show that oral administration of tributyrin has positive effects on the colonic restructuring in experimental colitis. In addition to reducing mucosal, damage and intestinal permeability, tributyrin supplementation causes several changes in the immune response.

Many of the animal studies using tributyrin in mice demonstrate a therapeutic effect using a standard dose of 5 mg/kg body weight. Using known, standard calculations, this corresponds to a human equivalent dose (HED) of 286 mg for a 70 kg person. Commercial dietary supplement consumer products recommend therapeutic dosages between 300 mg and 1,000 mg of tributyrin. The relationship between the HED of 286 mg from mice studies and a minimum human commercial product recommended dose of 300 mg is clear. The recommended human dose is typically rounded up. It is typical that higher doses may be recommended (if the minimum is good then more is better and a 70 kg person corresponds to 154 lb, while many people with health concerns are above this weight). Thus, there appears to be no specific suggestion or support for a lower human therapeutic dose for tributyrin as a butyrate generator. In fact, the lack of any support or suggestion for a lower dose indicates a lack of belief or knowledge that a lower dose is therapeutic, as there always is a commercial incentive for a lower dose-lower cost and easier to formulate into a dietary supplement.

The combination of tributyrin and probiotics and/or prebiotics is generally known. However, there is no suggestion or knowledge or support for any synergistic effect of combining probiotics and/or prebiotics with tributyrin leading to an increased production of butyrate to therapeutic levels.

Probiotics and prebiotics are known to convey health benefits on consumption. However, their use sometimes requires large quantities or doses of active ingredients. In addition, it is known that prebiotics and probiotics do not always convey similar health benefits to all mammals including humans. Many people are known as “non-responders” and do not achieve the purported health benefits from taking prebiotics/probiotics. It is believed that this may be partially due to the composition of their microbiome, which has limited ability to greater beneficial SCFAs like butyrate. It is believed that people with chronic diseases like cancer and auto-immune diseases, may have lowered ability to generate butyrate in the body. Tributyrin can therefore be used to supplement the endogenous generation of butyrate in these individuals and help bring them back to normal healthy butyrate levels.

A need therefore exists for new compositions of dietary supplements and supplement compositions with enhanced health benefits across a wider spectrum of mammals including humans, and for those with chronic diseases with lowered abilities to generate butyrate.

A need exists for a dietary supplement with improved effects on health. Specifically, a need exists for a dietary supplement that may be taken for the purpose of improving health as a replacement for probiotics and/or prebiotics for the generation of butyrate at therapeutic levels.

Moreover, a need exists for a dietary supplement for improving health incorporating tributyrin at a lower therapeutic dose resulting in improvements in the ability to formulate a dietary supplement and reduce the cost.

A need also exists for a dietary supplement where tributyrin is a replacement for a probiotic and/or prebiotic with enhanced health benefit. Tributyrin generates butyrate directly in the body and is thus not dependent on the state of, or the composition of, the microflora, i.e, the microbiome, of a specific individual.

A need also exists for a dietary supplement composition where tributyrin is combined with a prebiotic and/or postbiotic for enhanced health benefit. Prebiotics and probiotics may convey other health benefits, which may be separate from the generation of butyrate. Thus, a need exists for combinations of tributyrin with probiotics and/or prebiotics for enhanced generation of butyrate and the ability to convey other health benefits.

Moreover, a need further exists for a dietary supplement composition including a powder form of tributyrin for providing therapeutic generation of butyrate, where the powder form has a low water activity making it suitable for combining with probiotics and other moisture sensitive dietary ingredients

### SUMMARY

The present disclosure relates generally to dietary supplements and compositions administered in a therapeutically effective amount for effecting health through the generation of butyrate. Specifically, the present disclosure relates to novel dietary supplements and compositions wherein tributyrin is provided at a low therapeutic dose range, as a replacement for prebiotics and probiotics, and also for use in combination with prebiotics and probiotics. Prebiotics and probiotics activity in the gut cause the generation of butyrate. Butyrate is one of the short chain fatty acids believed to be responsible for energy generation and for signaling activities, which in turn effect the health of body organs and overall health.

In the present disclosure, tributyrin, which also generates butyrate in the gut, is designed to be a replacement for a prebiotic or a probiotic. Additionally, the present disclosure provides a lower therapeutic composition or dose range of tributyrin useful for the generation of butyrate than previously disclosed. Tributyrin can therefore be used as a direct replacement for a probiotic and a prebiotic but can also be used in conjunction with prebiotics and probiotics, as an alternative therapy to improve health. Thus, the present disclosure relates to a dietary supplement and composition comprising tributyrin used in a therapeutically effective amount for positively effecting health in a mammal. Furthermore, the use of tributyrin and the generation of butyrate is independent of the state or condition of the microbiome of the subject utilizing the present supplement.

The present disclosure further discloses a dietary supplement composition in a unique powder form with an extremely low water activity in order to better formulate the composition in combination with probiotics and other moisture sensitive dietary ingredients.

In one embodiment, the dietary supplement may be used for improving health, wherein the supplement includes a specific dosage of tributyrin in a dose range lower than what is generally known to produce therapeutic levels of butyrate in the gut of a mammal.

It is also an advantage and objective of the present disclosure to provide an improved dietary supplement for improving and enhancing health, wherein the dietary supplement is a replacement for probiotics and/or prebiotics.

It is yet another advantage and objective of the present disclosure to provide a dietary supplement composition, wherein a specific dosage of tributyrin in a dose range lower than what is generally known to produce therapeutic levels of butyrate is combined with either a probiotic or a prebiotic.

While multiple embodiments are disclosed, still other embodiments of the disclosure will become apparent to those skilled in the art from the following detailed description, which shows and describes illustrative embodiments of the disclosed compositions and methods. As will be realized, the disclosed compositions and methods are capable of modifications in various obvious aspects, all without departing from the spirit and scope of the disclosure. Accordingly, the detailed description is to be regarded as illustrative in nature and not restrictive.

### DETAILED DESCRIPTION

The present disclosure relates generally to dietary supplements and compositions containing tributyrin administered in therapeutically effective amounts for effecting health in a mammal. Disclosed are dietary supplements and compositions comprising therapeutically effective dosage amounts of tributyrin which are lower than previously known, for use in effecting health as related to the production of butyrate in the body at therapeutic levels, and other health benefits resulting from the generation of butyrate. The present disclosure also relates to tributyrin as a replacement for probiotics and/or prebiotics for enhanced health benefits as related to the production of butyrate in the body. The present disclosure further relates to dietary compositions of tributyrin in combination with either probiotics and/or prebiotics for enhanced synergistic health benefits as related to the production of butyrate in therapeutic amounts in the body and resulting health benefits. Finally, the administration of tributyrin as a dietary supplement and/or composition and the generation of butyrate and resulting benefits, is not dependent on the state or condition of the microbiome of the subject receiving the supplement.

### Methodology

In order to assess the action of tributyrin with and without prebiotics and probiotics, the present disclosure includes the use of two different model systems, which are described below. Currently, it is not possible to conveniently assess the production of butyrate in-situ, in the human body, or even in live animals. Therefore, the standard method for assessing both SCFA production and assessing the state and composition of the microbiome, is to measure and observe changes in the feces. Changes in the composition of feces provides an indicator of changes to the microbiome and gut.

Simply increasing bacteria in the microflora which are known to generate butyrate, in of itself, does not demonstrate a therapeutic benefit. An important aspect of the present disclosure is that the direct generation of butyrate provides a “therapeutic benefit” to the overall health of the subject mammal, including humans. In order to demonstrate the proposed “therapeutic benefit” of the generation of butyrate the present model systems compare butyrate generation in the gut to the generation of butyrate by known therapeutic prebiotics, such as inulin, provided at known therapeutic dosages. In this manner, the present disclosure demonstrates for the first time that tributyrin generates butyrate in amounts having a therapeutic benefit.

In the examples discussed below, three different prebiotics are used: (a) inulin, a well-known prebiotic which has been used therapeutically for gut health at a 4 g/day dose; XOS (xylooligosaccharide) a prebiotic derived from milk, with a demonstrated therapeutic benefit for gut health at 1 gram/day (a low dose prebiotic); and partially hydrolyzed guar gum (PHGG), with a therapeutic dose of 5-10 g/day. All three of these prebiotics are butyrate generators and all have demonstrated therapeutic benefits at these identified dose levels. In different examples, these known therapeutic prebiotics are used to compare against tributyrin butyrate generation in the present disclosure, and thus demonstrate a therapeutic benefit of the administration of tributyrin and the resulting generation of therapeutic amounts of butyrate.

In the first type of experiment, an ex-vivo model system is used. In this model system, an ex vivo simulation of the fermentation that occurs in the colon was used. Human feces from 6 volunteers were used as a gut microbiota starter cultures. The test materials were added to this system and butyrate generation was determined after 48 hours of fermentation, compared to controls for each set. Butyrate generation was measured using chromatography.

In this series of experiments the human equivalent dose (HED) was determined to be 1 gram/day of ingested prebiotic in-vivo corresponds to approximately 1 g/L in-vitro dose. Therefore a 10 mg/ml in-vitro dose is equivalent to 10 mg/day HED.

In the second type of experiment, an animal model system was used to study changes in the gut microbiome behavior. Caenorhabditis elegans (C. elegans), a nematode commonly used as an organism model, was used in the present study. Reduction of oxidative stress was determined in the C. elegans model. Butyrate has previously been shown in rodent and porcine studies to ameliorate oxidative stress in the intestine generated via hydrogen peroxide or Diquat dibromide exposure. Thus, an oxidative stress assay is a rapid indirect method of determining whether the tributyrin doses proposed in this study can produce beneficial butyrate in vivo.

Oxidative stress was induced in C. elegans by administering a lethal dose of 250 mM of the herbicide, Paraquat. Previous studies have shown that this concentration of Paraquat will kill adult C. elegans in liquid culture within 24 hours of exposure via the generation of superoxide anions that lead to oxidative damage of vital cellular components. The standard methods of growing the animals, treating with Paraquat, washing out the Paraquat, and observing survival at 12 hours post Paraquat treatment have been previously described. One group received only Paraquat as a positive control, while a second group received nothing as a negative control, and all others received the therapeutic treatments. At least 60 animals per group were used in the studies.

In the C. elegans series of studies, the human equivalent dose (HED) of tributyrin was determined in the following manner. A typical human stomach size of 250 ml was used as a baseline. 100 mg per serving per day of tributyrin for a 60 Kg adult is equivalent to 400 μg/ml (~1.32 mM). Tributyrin is soluble in DMSO up to a maximum concentration of 100 mg/ml, which is equivalent to 330.73 mM. C. elegans can be exposed to a 1% solution of DMSO in aqueous standard nematode growth media without compromising its viability. Doses between 10 mg HED [40 μg/ml (~0.13 mM)], and 100 mg HED [400 μg/ml (~1.32 mM)] were used.

### EXAMPLES

In addition to providing a method for determining a preferred therapeutic dose of tributyrin to effect health, the present disclosure also claims a novel therapeutic composition for tributyrin to effect health based on reduction to practice (Examples 1 and 2). In these examples, preferred human equivalent dosages of 10 to 100 mg per day were found to be y effective for generating therapeutic levels of butyrate and for enhancing health. An oxidative stress assay is a indirect method of determining whether the doses proposed in this study can produce beneficial butyrate in vivo (Example 3). Further, the present disclosure provides an enhanced or synergistic effect when tributyrin is combined with probiotics or prebiotics for the generation of butyrate (Example 4). The enhanced butyrate generation therapeutic effect demonstrated in these Examples is unique and not previously known.

### Example 1

In this study using the fecal sample model system, low doses of tributyrin were used demonstrating (a) butyrate generation at HED between 50 and 100 mg/day oil, (b) equivalence of butyrate generation for tributyrin to a HED of 4 g/day of a prebiotic, i.e., inulin (common therapeutic dose). Since the therapeutic dose of inulin is known to be about 4 grams per day this experiment shows that tributyrin at HED of 50 mg and 100 mg/day are therapeutically effective butyrate generation dosages. This level of therapeutic dose for butyrate generation has not been known or demonstrated previously.

| | | Human | Butyrate | |
|---|---|---|---|---|
| | Test | Equivalent | Generation | Standard |
| Test | Dose | Dose (per day) | (mM) | Deviation |
| Blank | | | 2.86 | 0.87 |
| Inulin | 4 g/L | 4 g | 3.62 | 1.52 |
| Tributyrin | 50 mg/L | 50 mg | 3.22 | 0.86 |
| Tributyrin | 100 mg/L | 100 mg | 3.73 | 0.88 |

The results from Example 1 show that low dose levels of tributyrin, between HED 50 and 100 mg/day generate about the same amount of butyrate as a therapeutic dose of inulin at an HED of 4 g/day. The differences in butyrate generation are not statistically different, but in all cases are statistically different from the blank (control).

### Example 2

In this example, the same fecal sample method was used to study butyrate generation compared with a prebiotic XOS at a HED of 1 g/day (lower end therapeutic usage level). XOS (xylooligosaccharide) is a prebiotic derived from milk and is one of the lower therapeutic dose prebiotics available on the market. Therapeutic doses of XOS have been described as high as 2.8 g/day so the choice of 1 g/day is at the extreme lower end of a suitable prebiotic dose.

In this experiment, the results show equivalent butyrate generation between 100 mg/day HED tributyrin and 1 g/day HED XOS, indicating a therapeutic generation level with 100 mg/day tributyrin. The results for tributyrin and XOS are statistically significantly different from the control (blank), again demonstrating the therapeutic generation of butyrate by tributyrin at low doses.

| | | Human | Butyrate | |
|---|---|---|---|---|
| | Test | Equivalent | Generation | Standard |
| Test | Dose | Dose (per day) | (mM) | Deviation |
| Blank | | | 2.78 | 0.67 |
| XOS | 1 g/L | 1 g/L | 3.30 | 0.64 |
| Tributyrin | 100 mg/L | 100 mg | 3.51 | 0.65 |

### Example 3

In this example, the C. elegans model as described above was used to measure oxidative stress as a health condition, and specifically as an indirect measurement of butyrate generation in the nematodes C. elegans. An oxidative stress assay is a rapid indirect method of determining whether the doses proposed in this study can produce beneficial butyrate in vivo. The results illustrated below show significant reduction in oxidative stress with tributyrin HED of 10 and 100 mg/day. PHGG was used to demonstrate the comparative therapeutic benefit. In this series of experiments, butyrate was not measured directly in the organisms, but PHGG is a known butyrate generator which generates therapeutic levels of butyrate at a PHGG dose of 5-10 g/day. Oxidative stress reduction was used as a measure of butyrate generation and compared tributyrin at different dosages to PHGG. The results show that those organisms provided with tributyrin at a HED of 10 and 100 mg/day, survived as well as those organisms provided with PHGG, thus demonstrating a correlation between the benefits of butyrate generation from the tributyrin compared to a known probiotic.

| | | Human | % Survival | |
|---|---|---|---|---|
| | | Dose | of Worms | |
| | | Equivalent | at 12 | Standard |
| Test | Test Dose | (per day) | hours | Deviation |
| Negative Control | | | 98.69 | 1.33 |
| Positive Control (Paraquat) | 250 nM | | 67.63 | 9.99 |
| PHGG | 20 mg/mL | 5 g | 96.24 | 2.56 |
| PHGG + Paraquat | 20 mg/mL + 250 nM | 5 g | 49.39 | 3.98 |
| PHGG | 40 mg/mL | 10 g | 96.27 | 0.82 |
| PHGG + Paraquat | 40 mg/mL + 250 nM | 10 g | 62.99 | 3.98 |
| Tributyrin | 40 ug/mL | 10 mg | 97.24 | 2.85 |
| Tributyrin + Paraquat | 40 ug/mL + 250 nM | 10 mg | 68.86 | 4.07 |
| Tributyrin | 400 ug/mL | 100 mg | 99.23 | 1.25 |
| Tributyrin + Paraquat | 400 ug/mL + 250 nM | 100 mg | 73.63 | 2.90 |

### Example 4

In this example, tributyrin is combined with two different probiotics in separate experiments specifically, Lacticaseibacillus rhamnosus GG (LGG) and Limosilactobacillus reuteri (REU). LGG is a gram-positive probiotic used to treat female urogenital tract infections and for the prevention of rotavirus diarrhea in children LGG is a known butyrate generator. REU is a lactic acid bacteria found in the GI tract and is used as a probiotic for the treatment of pediatric diarrheal disease. Although REU does not produce butyrate directly, it is capable of activating butyrate.

As in the other examples, the action of tributyrin (and in this case combined with probiotics), was compared with a prebiotic (XOS) with demonstrated therapeutic benefit. The results unexpectedly show a marked increase in butyrate generation versus tributyrin alone or the two probiotics-LGG and REU alone. The synergistic combination of tributyrin and each of the probiotics demonstrates an enhanced butyrate generation effect not previously observed. Coupled with the therapeutic dose of XOS, this example supports the assertion that tributyrin at a low dose, combined with a probiotic, produces enhanced butyrate generation with a therapeutic effect.

| | | Human | Butyrate | |
|---|---|---|---|---|
| | | Equivalent | Generation | Standard |
| Test | Test Dose | Dose | (mM) | Deviation |
| Blank | | | 2.78 | 0.67 |
| XOS | 1 g/L | 1 g | 3.30 | 0.64 |
| Tributyrin Oil | 100 mg/L | 100 mg | 3.51 | 0.65 |
| LGG | 5 * 10\hat{ }7 CFU/mL | 10\hat{ }7 CFU | 3.15 | 0.67 |
| Tributyrin Oil + LGG | 100 mg/L + 5*10\hat{ }7 | 100 mg + 10\hat{ }7 | 9.03 | 0.90 |
| REU | 5 * 10\hat{ }7 CFU/mL | 10\hat{ }7 CFU | 2.72 | 0.66 |
| Tributyrin Oil + REU | 100 mg/L + 5*10\hat{ }7 | 100 mg + 10\hat{ }7 | 9.63 | 0.76 |

### Powder Composition

Tributyrin is an oil. An oil form is suitable for applications such as with softgels and liquid capsules. However, the present disclosure also provides for a novel powder form of the present composition including tributyrin. This powder form contains partially hydrolyzed guar gum and other inert ingredients. Partially hydrolyzed guar gum is a prebiotic itself but has also been shown to improve absorption of active ingredients.

Unexpectedly, the addition of 10% partially hydrolyzed guar gum significantly lowered the water activity of the powder. The use of partially hydrolyzed guar gum for this purpose has not been previously known or described. The powder composition also contains acacia gum which has film forming properties, which is important for spray drying of powder. The powder composition contains between 30 and 70% tributyrin, so the quantity of powder used to meet the dose range for tributyrin is between 1.43×(70%) and 3.33×(30%) the amount of oil required. The powder form is suitable for other consumer formats such as capsules and tablets. Both the oil and powder forms are suitable for the therapeutic purpose provided in the present disclosure.

Two different tributyrin powder compositions were created as follows: Composition A contains 50% tributyrin oil, 27% acacia gum, 10% partially hydrolyzed guar gum, 2% rosemary extract, 1% microcrystalline cellulose. Composition B contains 50% tributyrin oil, 37% acacia gum, 2% rosemary extract, 1% microcrystalline cellulose. The water activity for Composition A was measured to be 0.12. The water activity for composition B was measured to be 0.30. A water activity of less than 0.2 is necessary and desirable for combination with probiotics and/or other moisture sensitive dietary ingredients to create the powder composition.

The previous discussions focus generally on compositions of tributyrin for adult human oral consumption. However, with suitable dose modifications. these compositions are also suitable for animal (pet, livestock, etc.) oral consumption and human child oral consumption for the enhanced generation of butyrate to improve health, using known conversions.

The above disclosure relates primarily to the use of tributyrin for use in adult humans. However, tributyrin compositions between 10 and 100 mg per day human dose, based on a human weight of 70 kg, can be converted into animal doses, for pet or livestock applications, using known or suitable conversion factors. In dogs, for example, an oral equivalent dose range is calculated at approximately 0.257 to 2.57 mg/kg dog weight. Tributyrin compositions between 10 and 100 mg per day human dose, based on a human weight of 70 kg, can be converted into children's equivalent doses of approximately 0.143 to 1.43 mg/kg child weight. A slightly modified version of Clark's rule was used for this calculation—Clark's rule is based on a 68 kg adult; the present disclosure uses a 70 kg adult for the calculations. Other potential factors, including age, health conditions and/or disease/illness, other medications being administered, may also play a role to determine the dosage amount of tributyrin

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

1-19. (canceled)

20. A method of synergistically increasing production of butyrate in the gut of a mammal, the method comprising the steps of:
preparing a dietary supplement composition comprising:
between about 30 and 70% tributyrin;
between about 5 and 15% partially hydrolyzed guar gum fiber; and
between about 15-65% other ingredients;
administering the composition to the mammal, wherein combination of the tributyrin with partially hydrolyzed guar gum fiber produces a therapeutically effective increase in the amount of butyrate in the gut of the mammal for improving health in the mammal; and,
wherein the composition is administered daily to the mammal so that the mammal receives a daily dosage of about 10-100 mg tributyrin and about 0.7-50 mg partially hydrolyzed guar gum fiber providing a total daily dose of about 14.3 to about 333.3 mg of the composition.

21. The method of increasing production of butyrate in the gut of a mammal dietary supplement composition of claim 20, wherein the composition has a water activity of less than 0.2.

22. The method of increasing production of butyrate in the gut of a mammal of claim 20, wherein the therapeutically effective production of the increased amount of butyrate in the gut of the mammal is independent of a composition of a microbiome in the gut.

23. The method of increasing production of butyrate in the gut of a mammal of claim 20, wherein the method further includes providing a loading dosage amount of 30% tributyrin and 5% to 15% partially hydrolyzed guar gum providing a range of about 33.3-333.3 mg per day of the composition.

24. The method of increasing production of butyrate in the gut of a mammal of claim 23, wherein the loading dosage amount provides a range of about 10-100 mg of tributyrin per day.

25. The method of increasing production of butyrate in the gut of a mammal of claim 24, wherein the loading dosage amount of about 10-100 mg of tributyrin is administered daily to a mammal based on a given weight of the mammal.

26. The method of increasing production of butyrate in the gut of a mammal of claim 20, wherein the method further includes providing a loading dosage amount of 70% tributyrin and 5% to 15% partially hydrolyzed guar gum providing a range of about 14.3-142.9 mg per day of the composition.

27. The method of increasing production of butyrate in the gut of a mammal of claim 26, wherein the loading dosage amount provides a range of about 10-100 mg of tributyrin per day.

28. The method of increasing production of butyrate in the gut of a mammal of claim 27, wherein the loading dosage amount of about 10-100 mg of tributyrin is administered daily to a mammal based on a given weight of the mammal.

29. The method of increasing production of butyrate in the gut of a mammal of claim 20, wherein the method further includes providing a loading dosage amount of 50% tributyrin and 5% to 15% partially hydrolyzed guar gum providing 200 mg per day of the composition.

30. The method of increasing production of butyrate in the gut of a mammal of claim 29, wherein the dosage amount provides 100 mg tributyrin.

31. A method of synergistically increasing production of butyrate in the gut of a mammal, the method comprising the steps of:
providing tributyrin in a dosage amount of between 10 and 100 mg per day;
providing partially hydrolyzed guar gum fiber provided in a dosage amount of between 0.7 and 50 mg per day;
combining the tributyrin with the partially hydrolyzed guar gum creating a composition providing a therapeutically effective increase in an amount of butyrate in the gastrointestinal tract of the mammal when the composition is administered daily to the mammal.
